(19) United States
(12) Patent Application Publication
(10) Pub. No.: US 2014/0142372 A1
(43) Pub. Date: May 22, 2014

(54) CHRONO-GASTRIC MODULATION

(71) Applicant: Marc Danan, Toulon (FR)

(72) Inventor: Marc Danan, Toulon (FR)

(21) Appl. No.: 13/681,502

(22) Filed: Nov. 20, 2012

(51) Int. Cl. A61F 5/00

(52) U.S. Cl. CPC A61F 5/0059
USPC 600/37

(57) ABSTRACT

An auto-regulated CGM (Chrono Gastric Modulation) that enables adjusting a patient's food intake through the day.

### RELATED APPLICATIONS

The present application claims priority from U.S. Provisional Patent Application 61/561,877, filed 20 Nov. 2011, which is incorporated herein by reference.

### FIELD OF THE INVENTION

The present invention relates generally to control of obesity, and particularly to a controllable gastric ring for chrono-gastric modulation.

### BACKGROUND OF THE INVENTION

Currently, within the framework of the surgical care of adult obesity, various surgical techniques exist, one of them being the Adjustable Gastric Ring (AGA) and which functions as follows:

Restrictive technique: The AGA is a restrictive technique which decreases the volume of the stomach and slows down the passage of food; it does not disrupt food digestion. A ring, with a modifiable diameter, is placed around the top of the stomach thus creating a small pocket; little food is then necessary to fill this pocket and the sensation of satisfaction quickly occurs. Food will very slowly flow according to the hourglass principle.

The only adjustable technique: The ring is connected by a small tube to a control case placed under the skin. This ring can be tightened or loosened by injecting a liquid in the case, through the skin. A radiological monitoring is necessary during follow-up.

The ring can be removed with a new procedure in case of complication, ineffectiveness or at the patient's request.

Limits of the gastric ring: Few aspects of the gastric ring, including problems with food tolerance, possible long term complications like esophageal dilation and weight loss failure, have put this technique under scrutiny.

There are two different types of outcome in AGA patients:
Patients who will perceive the limitation (low food tolerance, complications like esophageal dilation, weight loss failure) and:
Patients who won't perceive the limitation (absence of mechanical effect, esophageal flushing effect through the ring)

Food tolerance is low for the patients who do not feel satisfied, or despite a satisfied feeling, ignore it and continue eating therefore overeating which usually induces vomiting.

This tolerance is very variable throughout the day:
Intolerance in the morning
No perception of limitation in the evening

Esophageal dilation: This complication is largely due to the result of a rejection mechanism from the esophagi, due to an excessive alimentation or a gastric band set too tight. Indeed, loosening the gastric ring often leads to the disappearance of this dilation.

Weight loss failure: Mostly occurs because of misdiagnosed issues during follow-ups:
Dietary behavior issues: binging, not following the rules of food restrictions, excessive food division, increase in the energy density of portions.
Psychological issues: eating disorders of compulsive types.
Surgical issues: the ring is improperly installed and leads to a “non-satisfaction” feeling or ,the ring is too tight and prevents the patients from having a healthy diet which will result in consuming foods that are “easy to eat” like chocolate or soft cookies.

Other influencing factors:
Stress
Hormonal impregnation (pregnancy) or medicinal (corticoids)

Whether the size of the gastric ring is modifiable electronically or through the manual injection of a salty solution, this adjustment is only possible with the intervention of the practitioner.

New electronically monitored AGA devices are now emerging; their goal is to enable adjustments of the ring through a remote control device and replace the hydraulic technique.

Dietetic aspects: In order to avoid deficiencies and maintain the patient's health, it would be beneficial to allow a sufficient food bowl at meal times under a dietician' s supervision. From a strictly dietetic point of view, it would be beneficial to allow the patient a controlled but sufficient food portion at discrete times (like meal times) and avoid food intake when in it is not meal time.

Today, the patient is advised to fraction his food intake and minimize the volume of his/her meals to ease digestion and avoid unpleasant and painful “regurgitations”, these are usually the consequence of an excessive food volume in the gastric pocket, not enough time interval left between meals, insufficient mastication, etc.

A correct eating education, provided by a dietician, is essential but challenging since the patient is faced with the necessity of changing his/her eating habits both from a quantity/quality point of view as well as in a timely one.

### SUMMARY OF THE INVENTION

The present invention seeks to provide an improved controllable gastric ring for chrono-gastric modulation, as is described more in detail hereinbelow.

In the prior art, the patient cannot take a sufficient food portion (ring close) due to associated vomiting and pain. The CGM (Chrono Gastric Modulation) technology of the present invention will enable adjusting the patient's food intake through the day, and help the patient to have an educated, nutritional, beneficial diet.

Other physiological factors like neuro-hormonal mechanisms and esophageal innervations may also interfere with the maintenance of satisfaction, but the restrictive aspect of the prior art gastric band prevails. In contrast, in the present invention, the success of the gastric ring depends (among other things) on its adjustment ability, which is dynamic, not static, according to the principle of the “chrono gastric modulation” technology.

This dynamic adjustment of the invention, tailored to fit each patient after examination by the surgeon, dietician and psychologist, will be prompted by a software program.

There is thus provided in accordance with an embodiment of the present invention, a system for treating obesity including a ring positioned around the stomach of a user, and a controller using a timer, wherein the controller is programmed to tighten the ring around the user's stomach for a period of time calculated by the timer.

In accordance with an embodiment of the present invention, after the period of time, the controller is operative to loosen the ring around the user's stomach.

In accordance with an embodiment of the present invention, an analyzer is provided to analyze a quantity and/or quality of food consumed by the user so that the controller tightens the ring around the user's stomach for the period of time. The analyzer may be positioned on the user's esophagus. The controller may control the ring remotely. The controller may control the ring through Internet. The controller may be positioned under skin of the user.

### BRIEF DESCRIPTION OF THE DRAWINGS

The present invention will be understood and appreciated more fully from the following detailed description taken in conjunction with the drawings in which:

FIG. 1 is a simplified illustration of a CGM motorized ring and antenna, in accordance with an embodiment of the present invention.

FIGS. 2 and 3 are simplified illustrations of a CGM motorized ring and antenna and remote programming of the time switch by internet access, in accordance with further embodiments of the present invention.

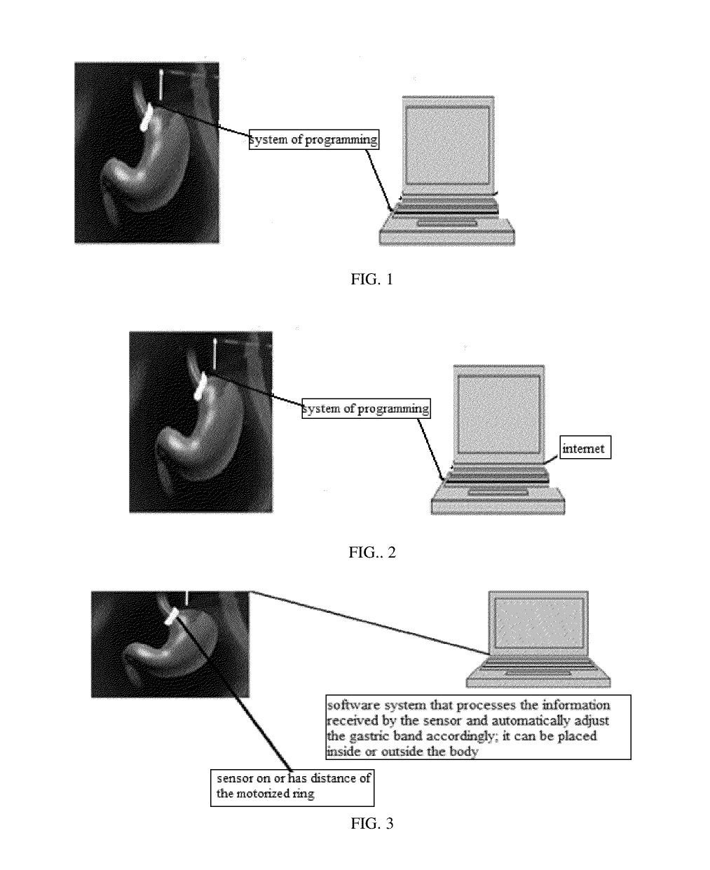

### DETAILED DESCRIPTION OF EMBODIMENTS

In accordance with a non-limiting embodiment of the present invention, there is a new electronically monitored AGA. The adjustment of the ring is through a remote control device. The embodiment is an auto-regulated CGM (Chrono Gastric Modulation). This technology will enable adjusting the patient's food intake through the day.

This dynamic adjustment, tailored to fit each patient after examination by the surgeon, dietician and psychologist, will be prompted by a software program.

The ring adjustment will be dynamic and not static.

The electric power-driven ring will open and close in a specific programmed timeframe controlled by the software.

The terms gastric band or gastric ring are used interchangeably.

A controlled study comparing two groups was used to evaluate this theory; the first group in the study has the gastric band adjusted in the current traditional way (hydraulically and with the intervention of the practitioner) and the second one has the CGM technology of the present invention implemented.

This study is possible thanks to the core feature of the CGM band which enables a remotely computerized monitoring through the creation of IT software (monitoring through the internet, mobile applications and auto-regulation)

This technology requires a motorized gastric band either with an embedded internal clock and rechargeable battery or a gastric band deprived of an internal clock or battery but with a command console, which can be internal (subcutaneous) or external.

The command console will be implemented with software capable of providing the amount of energy necessary to activate the motor of the ring.

AUTO-REGULATED CGM:

The principle is the same as for the CGM ring described above with an additional sensor placed either above the ring (at the level of the cardia) or within the ring itself.

This sensor is able to analyze the ingested food both quantitatively and qualitatively (proteins, carbohydrates, lipids) and automatically command the gastric band adjustment according to the collected data. In the case an excessive amount of calories ingested during the meal is detected, the band will contract even if the loosening time slot allowed for the patient is not over yet.

THE CGM

Function

The electric power-driven ring will open and close in a specific programmed timeframe controlled by the software.

Principle

The order to open and close the electric power-driven ring is sent to a command box that is either directly connected to the computer or remotely through an internet connection.

The motorized ring receives the information and responds accordingly by waves which are emitted by the box with an antenna.

SOFTWARE

The patient might not eat at the same exact time every day; for this reason he is given the ability to decide the starting time of his meal within the time frame allowed by the dietician.

For example, if it is decided that breakfast has to be taken between 7 am and 9 am, and that the “loosening” time slot is 15 minutes, the patient will be required to activate the start of the adjustment mechanism within the two hours timeframe whenever he is ready to have breakfast.

Parameters checked by the software

The “loosening” hours defined in hours and minutes

The time slot in minutes during which the ring will stay open

The number of time slot opening from 3 to 6

Example:

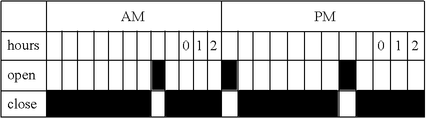

Auto-regulated CGM

Function

The power-driven ring will open and close in the time at the request of the software and according to the information received by the sensor.

The sensors: all sensors, existing or to come, aimed to serve the auto-commanded gastric ring technology, are in compliance with the scope of the Chrono Gastric Modulation.

Software

Additional functions correlated to the sensor functionality will be implemented within the specification of the CGM software discussed above.

EXTRAORDINARY SESSION

The patient can have an extraordinary session where the patient using a one time password will be able to access the software and command the adjustment of the ring in case of special situations.

What is claimed is:

1. A system for treating obesity comprising:
a ring positioned around the stomach of a user; and
a controller using a timer;
wherein the controller is programmed to tighten the ring around the user's stomach for a period of time calculated by the timer.

2. The system of claim 1, wherein after said period of time, said controller is operative to loosen said ring around the user's stomach.

3. The system of claim 1, further comprising an analyzer to analyze a quantity and/or quality of food consumed by the user so that the controller tightens the ring around the user's stomach for said period of time.

4. The system of claim 3, wherein the analyzer is positioned on the user's esophagus.

5. The system of claim 1, wherein the controller controls the ring remotely.

6. The system of claim 1, wherein the controller controls the ring through Internet.

7. The system of claim 1, wherein the controller is positioned under skin of the user.
